(19) United States
(12) Patent Application Publication
(10) Pub. No.: US 2004/0136908 A1
(43) Pub. Date: Jul. 15, 2004

(54) Anti-cd19 immunotoxins

(76) Inventors: William C. Olson, Issububg, NY (US); Paul J. Maddon, Scarsdale, NY (US); Dangshe Ma, Millwood, NY (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/474,469

(22) PCT Filed: Mar. 29, 2002
(86) PCT No.: PCT/US02/09889

(30) Foreign Application Priority Data
Sep. 4, 2001 (US) 60282587

(51) Int. Cl. A61K 51/00

(52) U.S. Cl. 424/1.49

(57) ABSTRACT

The invention relates to therapeutic methods using compositions including immunotoxins based on antibodies that specifically bind the B cell membrane protein CD19. Anti-CD19 immunotoxins, compositions containing such immunotoxins, and methods for using the immunotoxins are provided. Use of immunotoxins in the manufacture of medicaments for the treatment of various disorders also is provided.

### FIELD OF THE INVENTION

The invention provides therapeutic methods using compositions including immunotoxins based on antibodies that specifically bind the B cell membrane protein CD19.

### BACKGROUND OF THE INVENTION

B cell lymphomas constitute an important group of malignancies that include B cell non-Hodgkin's lymphoma (NHL), B cell acute lymphocytic leukemia (B-ALL), B cell precursor acute lymphocytic leukemia (pre-B-ALL), B cell chronic lymphocytic leukemia (B-CLL) and hairy cell leukemia. Non-Hodgkin's lymphomas comprise a heterogeneous group of lymphoid neoplasms that are predominantly B cell in origin. In the United States alone, approximately 240,000 people have B cell NHL and ˜60,000 new cases occur each year. The 5% annual increase in incidence is the fastest for any human cancer and is due in part to the increase in AIDS-associated lymphomas.

Therapeutic interventions for B cell malignancies include chemotherapy and radiation therapy. Although response rates are high, cure is rare and the median duration of response is only 2-3 years (Homing, Seminars in Oncol., 25 [Suppl]:75-88, 1993). There is an urgent need for new and less toxic therapies to prevent or combat disease relapse.

An antibody therapy (Rituxan™, U.S. Pat. No. 5,736,137, incorporated by reference herein) was recently approved by the United States Food and Drug Administration (FDA) for the treatment of relapsed or refractory low-grade or follicular, CD20-positive B-cell non-Hodgkin's lymphoma. Rituxan™ is a chimeric mouse-human monoclonal antibody to human CD20 (Genbank accession number X07203), a 35 kilodalton, four transmembrane-spanning protein found on the surface of the majority of B cells in peripheral blood and lymphoid tissue. In addition, lymphoma therapies employing radiolabeled anti-CD20 antibodies have been described in U.S. Pat. Nos. 5,595,721, 5,843,398, 6,015,542, and 6,090,365.

CD19 (Genbank accession number M28170) is a 95 kilodalton integral membrane glycoprotein present on cells of the B lineage. Several properties of the CD19 antigen make it a promising target for immunotherapy. CD19 is perhaps the most ubiquitously expressed antigen in the B cell lineage and is expressed on >95% of B cell lymphomas, including B-ALL cells that do not express CD20. CD19 is not expressed on pluripotent CD34+hematopoietic stem cells, and thus the B lineage can be repopulated following CD19-directed therapies. CD19 is also not expressed on terminally differentiated plasma cells or typical B cell myelomas, although there is evidence that these cells may derive from a transformed precursor cell that does express CD19 (Scheuermann and Racila, Leuk Lymphoma 18:385-397, 1995 and references therein). In addition, CD19 is expressed on few if any other cell types, which thus may be spared by CD19-directed therapies. CD19 is not shed into the circulation. Notably, CD19 expression is maintained on B cell lymphomas that become resistant to anti-CD20 therapy (Davis et al., Clinical Cancer Research, 5:611, 1999).

One CD19 immunotherapeutic has advanced into Phase III testing. This agent comprised a murine anti-CD19 antibody (B4) conjugated to a modified form of ricin, a plant toxin. In multiple Phase I and Phase II studies of this agent, objective responses were seen in a number of patients with tolerable and reversible toxicities. However testing was halted during Phase III testing due to issues related to the generation of immune responses to the murine antibody and to the toxin as well as a side effect known as vascular leak syndrome that is characteristic of the plant-based toxin (Monoclonal Antibody-Based Therapy of Cancer, M. L. Grossbard, editor, Marcel Dekker, New York, 1998 and references therein).

Other reports of the use of anti-CD19 antibodies have stated that the antibodies are ineffective in the treatment of B cell malignancies. Illidge et al. (Blood, 94:233-243, 1999) investigated radioimmunotherapy (RIT) of B-cell lymphoma (BCL) with radiolabeled monoclonal antibodies to B cell markers (anti-CD19, anti-CD22, anti-MHCII, and anti-Id). The results demonstrated that anti-CD19 and anti-CD22 were not active in therapy, whether administered to BCL-bearing mice as a radiolabeled antibody or as a naked antibody. The authors comment that unlabeled anti-CD19 monoclonal antibodies were previously shown by them to be therapeutic in B-cell lymphoma, but only when given in high amounts and for an extended period of time. The authors concluded that B-cell surface antigens expressed at a comparatively low level (CD19, CD22) were less suitable targets for RIT than more strongly expressed antigens (MHCII, Id). An additional conclusion is that antigens that are not endocytosed are superior targets for RIT. Accordingly, Illidge et al. teach that CD19 is an unsuitable target for radioimmunotherapy in view of its low expression level and rapid endocytosis upon ligand binding.

U.S. Pat. No. 5,686,072 disclosed the possibility of using high doses of unlabeled anti-CD19 antibodies (500 μg/mouse) in combination anti-CD22 immunotoxin in the treatment of lymphoma in mice. The treatment described in this patent, however, reflected the combination of the toxic activities of the anti-CD22 immunotoxin and a growth inhibition effect of the unlabeled anti-CD19 antibody; mice were not cured by either the immunotoxin alone or by anti-CD19 antibody alone, even at very high doses of antibody (5 mg/mouse).

Thus the use of CD19 antibodies alone or as an immunotoxin has, to date, been unsuccessful due to unacceptable side effects, toxicities, requirement for massive doses of antibody and/or a lack of effectiveness in treating B cell malignancies. Although the expression profile of CD19 appears to be suitable to the development of immunotoxin agents, such agents have not been successfully made or used.

Accordingly, there remains a need for immunotoxin having selectivity for malignant B cells and terminally differentiated B cells (but not hematopoietic stem cells), with an acceptable toxicity profile.

### SUMMARY OF THE INVENTION

Anti-CD19 immunotoxins, compositions containing such immunotoxins, and methods for using the immunotoxins have been identified that unexpectedly do not suffer from the deficiencies in the immunotoxins of the prior art.

According to one aspect of the invention, methods for treating a B cell malignancy in a subject are provided. The methods include administering to a subject in need of such treatment an amount of a composition comprising an anti-CD19 immunotoxin and a pharmaceutically acceptable carrier effective to treat the B cell malignancy.

In some embodiments the anti-CD19 immunotoxin is labeled with a cytotoxic radionuclide or radiotherapeutic isotope, such as an alpha-emitting isotope, a beta-emitting isotope, or an isotope that emits Auger and low energy electrons. Preferably the alpha-emitting isotope is selected from the group consisting of ²²⁵Ac, ²¹¹At, ²¹²Bi, ²¹³Bi, ²¹²Pb, ²²⁴Ra, and ²²³Ra. Preferably the beta-emitting isotope is selected from the group consisting of ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, and ⁶⁴Cu. Preferably the isotope that emits Auger and low energy electrons is selected from the group consisting of ¹²⁵I, ¹²³I and ⁷⁷Br.

In other embodiments the composition is administered intravenously.

In still other embodiments, the amount of the anti-CD19 immunotoxin administered to the subject is between about 10 μg/kg and about 100,000 μg/kg. Preferably the amount of the anti-CD19 immunotoxin administered to the subject is between about 100 μg/kg and about 10,000 μg/kg.

In certain embodiments the anti-CD19 immunotoxin includes a radionuclide and the amount of the radionuclide administered to the subject is between about 0.001 mCi/kg and about 10 mCi/kg. In some preferred embodiments, the amount of the radionuclide administered to the subject is between about 0.1 mCi/kg and about 1.0 mCi/kg. In other preferred embodiments, the amount of the radionuclide administered to the subject is between about 0.005 mCi/kg and 0.1 mCi/kg.

In other embodiments, the anti-CD19 immunotoxin comprises a monoclonal anti-CD19 antibody or antigen-binding fragment thereof. Preferably the monoclonal anti-CD19 antibody is a human monoclonal antibody, or a humanized monoclonal antibody, or is selected from the group consisting of B4, HD37, BU12, 4G7, J4.119, B43, SJ25C1, and CLB-CD19 antibodies.

In certain methods, the B cell malignancy is selected from the group consisting of B cell non-Hodgkin's lymphoma (NHL), B cell acute lymphocytic leukemia (B-ALL), B cell precursor acute lymphocytic leukemia (pre-B-ALL), B cell chronic lymphocytic leukemia (B-CLL) and hairy cell leukemia. In other embodiments, the B cell malignancy comprises B cells that do not express CD20.

In other embodiments, the methods further include administering to the subject one or more immunomodulatory agents, preferably a cytokine or an adjuvant. Preferred cytokines are selected from the group consisting of interleukin-1 (IL-1), IL-2, IL-3, IL-12, IL-15, IL-18, G-CSF, GM-CSF, thrombopoietin, and γ-interferon. The invention also includes embodiments in which one or more non-anti-CD19 immunotoxin therapies are administered to the subject, such as chemotherapy or radiation therapy.

In yet other embodiments, the anti-CD19 immunotoxin is labeled with a chemical toxin or chemotherapeutic agent. Preferably the chemical toxin or chemotherapeutic agent is selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

In further embodiments, the anti-CD19 immunotoxin is labeled with an agent that acts on the tumor neovasculature or an immunomodulator. Preferably the agent that acts on the tumor neovasculature is selected from the group consisting of combrestatin A4, angiostatin and endostatin. Preferably the immunomodulator is selected from the group consisting of α-interferon, γ-interferon, and tumor necrosis factor alpha (TNFα).

In another aspect of the invention, the anti-CD19 immunotoxins administered in the methods described above are provided.

In still another aspect of the invention, compositions that include the anti-CD19 immunotoxins administered in the methods described above and a pharmaceutically acceptable carrier are provided. Preferably the compositions are formulated for intravenous administration.

In yet another aspect of the invention, methods for treating an autoimmune disorder in a subject are provided. The methods include administering to a subject in need of such treatment an amount of the foregoing anti-CD19 immunotoxin compositions effective to treat the autoimmune disorder. Autoimmune disorders include plasma cell disorders including IgM polyneuropathies, immune thrombocytopenias, and autoimmune hemolytic anemias; Sjogren's syndrome; multiple sclerosis; rheumatoid arthritis; autoimmune lymphoproliferative syndrome (ALPS); sarcoidosis; diabetes; systemic lupus erythematosus; and bullous pemphigoid.

According to a further aspect of the invention, methods for deleting CD19⁺ B cells to reduce antibody formation in a subject are provided. The methods include administering to a subject in need of such treatment an amount of the foregoing anti-CD19 immunotoxin compositions effective to reduce antibody formation. In these methods, the composition can be administered before, during or after treatment for xenograft or transplantation processes.

The immunotoxins also are useful in the preparation of medicaments, particularly for B cell malignancies, autoimmune disorders, and transplantation.

According to still another aspect of the invention, use of the foregoing immunotoxins and compositions for the preparation of medicaments is provided. The medicaments are useful for the treatment of disorders caused by cells that express CD19, such as B cell malignancies, autoimmune disorders, and transplantation rejection. The medicaments also are useful for depleting or reducing B cells in a subject.

These and other aspects of the invention are described below.

### DETAILED DESCRIPTION OF THE INVENTION

Because CD19 is rapidly internalized upon antibody binding, it has been largely overlooked as a target for conventional radioimmunotherapies that employ ¹³¹I, which can be catabolized intracellularly and subsequently released into the circulation. However, antigen internalization potentiates other forms of immunotherapy, such as those that utilize metallic radionuclides or chemical toxins. CD19 thus represents a preferred target for these modes of therapy.

Accordingly, the invention provides anti-CD19 immunotoxins and methods for treating subjects having a B cell malignancy or B cell hyperproliferative disease by administering effective amounts of the anti-CD19 immunotoxins to the subjects. Preferably the anti-CD19 immunotoxins are radiolabeled with alpha emitter radionuclides or chemical toxins. Immunotoxins labeled with plant toxins are not preferred due to the side effects that typically accompany the administration of plant toxins such as ricin, as described above (e.g., vascular leak syndrome).

As used herein, the term “immunotoxin” refers to a conjugate comprising an antibody, or antigen-binding fragment thereof, conjugated to one or more toxin molecules. An anti-CD19 antibody includes an anti-CD19 antibody or antigen-binding fragment thereof. Various anti-CD19 antibodies are contemplated to be of use in accordance with the present invention, including, for example, B4, HD37, BU12, 4G7, J4.119, B43, SJ25C1, and CLB-CD19 (see, e.g., Nadler et al., J. Immunol. 131(1):244-50, 1983; Pezzutto et al., J. Immunol. 138(9):2793-9, 1987; Flavell et al., Br. J. Cancer 72(6) 1373-9, 1995; Bejcek et al., Cancer Res. 55(11):2346-51, 1995; Gunther et al., Leuk Lymphoma 22(1-2):61-70, 1996; Li et al., Cancer Immunol. Immunother. 47:121-30, 1998; Myers et al., Leuk Lymphoma 29:329-38, 1998; Chen et al., J. Clin. Pharmacol 39:1248-55, 1999; Vlasveld et al., Cancer Immunol. Immunother. 40:3747, 1995). Alternatively, one may generate other anti-CD19 antibodies using the monoclonal antibody technology which is generally known to those of skill in the art and described herein. In preferred embodiments, the anti-CD19 antibody generated is a fully human monoclonal antibody.

The invention, therefore, embraces antibodies or fragments of antibodies having the ability to selectively bind to CD19. As used herein, “antibody” includes both naturally occurring and non-naturally occurring antibodies. Specifically, “antibody” includes polyclonal and monoclonal antibodies, and monovalent and divalent fragments thereof.

Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man. Antibodies are prepared according to conventional methodology.

Monoclonal antibodies may be generated using the method of Kohler and Milstein (Nature, 256:495, 1975). To prepare anti-CD19 monoclonal antibodies useful in the invention, a mouse or other appropriate host animal is immunized at suitable intervals (e.g., twice-weekly, weekly, twice-monthly or monthly) with human CD19 antigen in the form of human B cells, B cell membranes, recombinant CD19, and/or CD19 protein purified from human B cells. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of CD19 by multiple routes.

Following the immunization regimen, lymphocytes are isolated from the spleen, lymph node or other organ of the animal and fused with a suitable myeloma cell line using an agent such as polyethylene glycol to form a hybridoma. Following fusion, cells are placed in media permissive for growth of hybridomas but not the fusion partners using standard methods, as described (Goding, Monoclonal Antibodies: Principles and Practice: Production and Application of Monoclonal Antibodies in Cell Biology, Biochemistry and Immunology, 3^{rd} edition, Academic Press, New York, 1996).

Following culture of the hybridomas, cell supernatants are analyzed for the presence of antibodies of the desired specificity, i.e., that selectively bind CD19 and B cells. Suitable analytical techniques include ELISA, flow cytometry, immunoprecipitation, Biacore (surface plasmon resonance), and western blotting. Other screening techniques are well-known in the field. Preferred techniques are those that confirm binding of antibodies to conformationally intact, natively folded CD19, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of anti-CD19 antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody, i.e., in the present invention, the ability to bind. CD19. However, using certain methods of humanization, the affinity and/or specificity of binding of the antibody for CD19 may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

Monoclonal antibodies may be produced by mammalian cell culture in hydridoma or recombinant cell lines such as Chinese hamster ovary cells or murine myeloma cell lines. Such methods are well-known to those skilled in the art. Bacterial, yeast, and insect cell lines can also be used to produce monoclonal antibodies or fragments thereof. In addition, methods exist to produce monoclonal antibodies in transgenic animals or plants (Pollock et al., J. Immunol. Methods, 231:147, 1999; Russell, Curr.Top. Microbiol. Immunol. 240:119, 1999).

An antibody can be linked to a detectable marker, an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra, or ²²³Ra. Alternatively, the cytotoxic radionuclide may a beta-emitting isotope such as ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, or ⁶⁴Cu. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of duocarmycin (see e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Toxins that are less preferred in the compositions and methods of the invention include poisonous lectins, plant toxins such as ricin, abrin, modeccin, botulina and diphtheria toxins. Of course, combinations of the various toxins could also be coupled to one antibody molecule thereby accommodating variable cytotoxicity. Other chemotherapeutic agents are known to those skilled in the art.

Agents that act on the tumor neovasculature can include tubulin-binding agents such as combrestatin A4 (Griggs et al., Lancet Oncol. 2:82, 2001) and angiostatin and endostatin (reviewed in Rosen, Oncologist 5:20, 2000, incorporated by reference herein). Immunomodulators suitable for conjugation to anti-CD19 antibodies include α-interferon, γ-interferon, and tumor necrosis factor alpha (TNFα).

The coupling of one or more toxin molecules to the anti-CD19 antibody is envisioned to include many chemical mechanisms, for instance covalent binding, affinity binding, intercalation, coordinate binding, and complexation. The toxic compounds used to prepare the anti-CD19 immunotoxins are attached to the antibodies or CD19-binding fragments thereof by standard protocols known in the art.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In preferred embodiments, it is contemplated that one may wish to first derivatize the antibody, and then attach the toxin component to the derivatized product. Suitable cross-linking agents for use in this manner include, for example, SPDP (N-succinimidyl-3-(2-pyridyldithio)propionate), and SMPT, 4-succinimidyl-oxycarbonyl-α-methyl-α (2-pyridyldithio)toluene.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the dithylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed ρ-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as ²²⁵Ac. Yet another example is DOTA (1,4,7,10-tetraazacyclododecane N,N′,N″,N′″-tetraacetic acid), which is a bifunctional chelating agent (see McDveitt et al., Science 294:1537-1540, 2001) that can be used is a two-step method for labeling followed by conjugation (see Example 4).

The invention also provides a method of treating a subject afflicted with a B cell malignancy, which comprises administering to the subject an effective amount of the anti-CD19 imnnunotoxin compositions described herein. As used herein, “subject” means any animal afflicted with a B cell malignancy. In preferred embodiments, the subject is a human. As used herein, “treating” means either slowing, stopping or reversing the progression of a B cell malignancy. Other clinical parameters may also be used to evaluate efficacy of treatment as are known by the skilled clinician such as increased survival time, inhibition of metastasis, and the like. In preferred embodiments, “treating” means reversing the progression to the point of eliminating the disorder. As used herein, “afflicted with a B cell malignancy” means that the subject harbors at least one cancerous cell that expresses B cell markers, including but not limited to CD19.

Thus the present invention has direct utility in the clinical treatment of various human diseases and disorders in which neoplastic B cells play a role. In particular, such B cell malignancies include B cell non-Hodgkin's lymphoma (NHL); B cell acute lymphocytic leukemia (B-ALL); B cell precursor acute lymphocytic leukemia (pre-B-ALL); B cell chronic lymphocytic leukemia (B-CLL); hairy cell leukemia; precursor B-lymphoblastic leukemia/lymphoma; prolymphocytic leukemia; small lymphocytic lymphoma; lymphoplasmacytoid lymphoma; immunocytoma; mantle cell lymphoma; follicular follicle center lymphoma; marginal zone B-cell lymphomas including extranodal (MALT-type+/− monocytoid B cells) and nodal (+/− monocytoid B cells); splenic marginal zone lymphoma (+/− villous lymphocytes); hairy cell lymphoma; plasmacytoma; plasma cell myeloma; large B-cell lymphomas including primary mediastinal (thymic) B-cell lymphoma; and Burkett's lymphoma.

Appropriate therapeutic regimens for using the present anti-CD19 immunotoxins will be known to those of skill in the art. Treatment may include administration of unlabeled anti-CD19 antibody prior to administration of anti-CD19 immunotoxin in order to block CD19 molecules on noncancerous cells. Methods of pre-treating with unlabeled antibodies to other tumor targets are described in U.S. Pat. No. 5,595,721.

The methods and compositions of the present invention are also contemplated to be of use in further clinical embodiments such as, for example, in the deletion or depletion of CD19+ B cells or a reduction in number of CD19+ B cells which produce undesirable or deleterious antibodies. Such undesirable or deleterious antibodies arise in autoimmune disorders and in xenograft or transplantation processes. Autoimmune disorders include plasma cell disorders including IgM polyneuropathies, immune thrombocytopenias, and autoimmune hemolytic anemias; Sjogren's syndrome; multiple sclerosis; rheumatoid arthritis; autoimmune lymphoproliferative syndrome (ALPS); sarcoidosis; diabetes; systemic lupus erythematosus; and bullous pemphigoid. In treating such disorders, the anti-CD19 immunotoxins of the invention are administered to the patient in amounts effective to delete, deplete or reduce the number of CD19+ expressing B cells and thereby diminish, reduce or eliminate detrimental antibody formation such as autoantibodies and the like. It is contemplated that doses representing effective amounts for this therapeutic purpose would be similar to the effective amounts described herein for the treatment of B cell malignancies.

It will be understood that the anti-CD19 immunotoxins of the invention may be administered alone, in combination with each other, and/or in combination with other therapies, such as chemotherapy and radiation therapy [see McLaughlin, et al., Semin. Oncol. 27(6 Suppl 12):37-41, 2000]. The anti-CD19 immunotoxins of the invention also may be cross-linked with other anti-tumor antibodies, such as anti-CD3, in heterodimeric diabodies (see Cochlovius et al., J. Immunol. 165(2):888-95, 1990).

Antineoplastic compounds that can be used in combination with the immunotoxins disclosed herein include, but are not limited to, the following sub-classes of compounds. Determination of dosages of antineoplastic compounds to be administered in combination with anti-CD19 immunotoxins for particular cancers is well within routine experimentation for one of ordinary skill in the art.

Antineoplastic agents include: Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gleevec; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta- I a; Interferon Gamma- I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab (Rituxan); Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives (e.g., 10-hydroxy- camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R═H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirirmostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O 6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer Supplementary Potentiating Agents include: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Antiproliferative agent: Piritrexim Isethionate.

Angiogenesis inhibitors: Endostatin, angiostatin, soluble troponin I.

Radioactive agents include: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Treatment may include administration of anti-CD19 immunotoxins with or without adjunct therapy. The adjunct therapy can include immunostimulatory or immunomodulatory agents. The immunomodulatory agent may include cytokines such as interleukins including IL-1, IL-2, IL-3, IL-12, IL-15, and IL-18; colony stimulating factors including G-CSF and GM-CSF; thrombopoietin, and interferons including γ-interferon. The immunomodulatory agent may be an immunologic adjuvant. The immunologic adjuvant also may comprise oligonucleotides containing unmethylated CpG dinucleotide sequences.

When administered, the therapeutic compositions of the present invention can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral or parenteral such as, intravenous, intraperitoneal, intramuscular, subcutaneous, intracavity, intranodal, intratumor, intrasynovial, transdermal, and the like. When antibodies are used therapeutically, a preferred route of administration is intravenous or by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resort to undue experimentation. When using antisense preparations of the invention, slow intravenous administration is preferred.

The compositions of the invention are administered in effective amounts. An “effective amount” is that amount of a anti-CD19 immunotoxin composition that alone, or together with further doses, produces the desired response, e.g. treats a B cell malignancy in a subject. This may involve only slowing the progression of the disease temporarily, although more preferably, it involves halting the progression of the disease permanently. This can be monitored by routine methods. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of anti-CD19 immunotoxins for producing the desired response in a unit of weight or volume suitable for administration to a patient. The response can, for example, be measured by determining the physiological effects of the anti-CD19 immunotoxin composition, such as regression of a tumor or decrease of disease symptoms. Other assays will be known to one of ordinary skill in the art and can be employed for measuring the level of the response.

The doses of anti-CD19 immunotoxins administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses can range from about 10 μg/kg to about 100,000 μg/kg. Based upon the composition, the dose can be delivered continuously, such as by continuous pump, or at periodic intervals. Desired time intervals of multiple doses of a particular composition can be determined without undue experimentation by one skilled in the art. Other protocols for the administration of anti-CD19 immunotoxin compositions will be known to one of ordinary skill in the art, in which the dose amount, schedule of administration, sites of administration, mode of administration and the like vary from the foregoing.

In general, doses of radionuclide delivered by the anti-CD19 immunotoxins of the invention can range from about 0.001 mCi/Kg to about 10 mCi/kg. In some preferred embodiments the dose of radionuclide ranges from about 0.1 mCi/Kg to about 1.0 mCi/kg. In other preferred embodiments, the dose of a radionuclide (e.g., an alpha-emitter radionuclide such as ²²⁵Ac) ranges from about 0.005 mCi/kg and 0.1 mCi/kg.

The optimal dose of a given isotope can be determined empirically by simple routine titration experiments well known to one of ordinary skill in the art.

Administration of anti-CD19 immunotoxin compositions to mammals other than humans, e.g. for testing purposes or veterinary therapeutic purposes, is carried out under substantially the same conditions as described above.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

An anti-CD19 immunotoxin composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of anti-CD19 immunotoxins, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diiuent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administration can be found in Remington 's Pharmaceutical Sciences, Mack Publishing Co., Easton, Pa.

### EXAMPLES

### Example 1

### Radiolabeling of Antibodies

Anti-CD19 antibodies are radiolabeled to attach a cytotoxic radionuclide to the antibody. Numerous monoclonal antibodies to CD19 are commercially available. For example, the B4 antibody is available in both IgG1 and IgG2a forms from Beckman-Coulter, Inc. (Miami, Fla.), as is the anti-CD19 antibody designated J4.119. The respective catalog numbers are 6602683, 6603708, and IM1283. Other anti-CD19 antibodies can be obtained using the methods described above.

A variety of technologies exist for attaching cytotoxic radionuclides to antibodies or antibody fragments (Magerstadt, Antibody Conjugates and Malignant Disease, CRC Press, Boca Raton, Fla., 1991). The method selected depends in part upon the nature of the radionuclide. Non-metallic radionuclides such as ¹³¹I can be linked directly to proteins, whereas chemical linkers are generally used with metallic isotopes such as ⁹⁰Y and ²¹³Bi.

By way of example, Na¹³¹I (PerkinElmer Life Sciences, Inc.) is oxidized using the chloramine T method or Iodogen (Pierce Chemical). The oxidized halide and protein of interest are combined according to the manufacturer's instructions. Ratios of 1 mCi isotope per 200 μg protein have been used successfully, but other ratios can be used to vary the specific activity of the radiolabeled protein. Following an appropriate incubation period, radiolabeled protein is separated from free isotope by size exclusion chromatography in the presence or absence of a suitable carrier protein, such as human serum albumin, or any other appropriate method. In this method, the halide can be attached to the protein of interest via an electrophilic substitution reaction on an aromatic amino acid such as tyrosine.

The chiral DPTA derivative 2-(4-isothicyanatobenzyl) diethylenetriamine pentaacetic acid (SCN—CHX-A″-DTPA) is conjugated to antibodies using previously described methods and apparatus (Nikula et al., Nucl. Med. Bio. 22:287, 1995; McDevitt et al. J Nucl. Med. 40:1722, 1999; Nikula et al., J. Nucl. Med. 40:166, 1999). In the following description, all buffers are prepared using metal-free water. As an added precaution, the buffers are passed over a Chelex-100 (BioRad Laboratories, Hercules, Calif.) ion exchange chromatography resin to further remove residual metals.

The B4 antibody is first rendered metal-free by dialysis or diafiltration against an appropriate buffer (e.g., 10 mM HEPES, 150 mM sodium chloride, pH 8.6) containing EDTA at 1-10 mM. The antibody is then dialyzed or diafiltered against buffer in the absence of EDTA. The antibody is then contacted with a molar excess of SCN—CHX-A″-DTPA overnight at ambient temperature. SCN—CHX-A″-DTPA is added in a 10- to 100-fold molar excess. Other ratios can be used in order to vary the degree of substitution. The conjugated antibody is then separated from unconjugated bifunctional chelator by further dialysis or diafiltration against a suitable buffer, such as 20 mM sodium acetate, 150 mM sodium chloride, pH 6.7. Parameters such as buffer pH, buffer identity, reaction time, reaction temperature, and chelator: antibody ratio can be varied in order to identify reaction conditions that are optimal for a given antibody.

The concentration of the immunoconjugate is determined by UV absorbance at a wavelength of 280 nm. The average number of chelates per antibody is determined by the yttrium arsenazo spectrophotometric method (Pippin et al., Bioconjug. Chem. 3:342-345, 1992). Typical conjugation ratios are 1-10 chelators per antibody. The optimal conjugation ratio can vary from antibody-to-antibody but can be determined empirically.

CHX-A″-DTPA-conjugated antibodies can be efficiently labeled with radiometallic isotopes such as ¹¹¹In and ⁹⁰Y, and ²¹³Bi. For ¹¹¹In or ⁹⁰Y, carrier-free isotope (PerkinElmer Life Sciences) is buffered to pH 4.5 with 3 M ammonium acetate. The anti-oxidant 1-ascorbic acid is added to a final concentration of 5 g/L as a radioprotectant. The isotope is typically combined with the immunoconjugate at a ratio of approximately 1-100 mCi/milligram, but other ratios can be used depending on the specific activity desired. The mixture is incubated at ambient temperature for 10-30 minutes. The reaction is quenched by the addition of a molar excess of EDTA. Radiolabeled antibody is separated from free isotope by passage over a 10 DG size exclusion chromatography column (BioRad Laboratories, Hercules, Calif.) using an acceptable mobile phase, such as 1% HSA. The immunoconjugates can be labeled with ²¹³Bi, ²²⁵Ac, or ¹⁷⁷Lu using similar methods as described previously (McDevitt et al., Applied Radiat.Isot., 50:895, 1999; Sgouros et al., J Nucl. Med., 40:1935, 1999; McDevitt et al., J Nucl. Med., 40:1722, 1999).

For the alpha particle, ²²⁵Ac, the bifunctional version of the chelating moiety DOTA (1,4,7,10-tetraazacyclododecane-1,4,7,10-tetraacetic acid) may be used to stably bind ²²⁵Ac to the antibody as described in McDevitt et al., Science 294:1537-1540, 2001.

### EXAMPLE 2

### In vitro Testing of Radiolabeled Antibodies

The immunoreactivity of the radiolabeled antibody is determined as described (McDevitt et al., J Nucl. Med. 40:1722, 1999) using a CD19-positive human B cell line such as Ramos, Daudi, Raji or Namalwa. Each of these cell lines is available from the American Type Culture Collection (Catalog numbers CRL-1596, CCL-213, CCL-86 and CRL-1432, respectively). CD19-negative human T cell lines such as MOLT-4 or Sup-T1 (ATCC Catalog numbers CRL-1582 and CRL-1942, respectively) are used as negative controls. The reaction yield and radiochemical purity of purified product are determined using instant thin layer chromatography and size exclusion high pressure liquid chromatography as described (McDevitt et al., J Nucl. Med. 40:1722, 1999). Because the chelation chemistries of ⁹⁰Y and ¹¹¹In are similar, the gamma-emitting isotope can be substituted for ⁹⁰Y for ease of detection in the in vitro studies.

Antibody-induced internalization of CD19 is measured by incubating radiolabeled antibody at a suitable concentration (e.g., 0.1-1 mg/ml) with ˜5×10⁴ CD19-positive human B cells (e.g., Raji, Ramos, Namalwa) for ˜2 hr at 37° C. in serum-containing medium. This incubation period can be varied to determine the kinetics of internalization. Cells are pelleted by centrifugation and then washed with media. Surface-bound radiolabeled antibody is stripped with pH 2.8 glycine buffer at ambient temperature for approximately 10 minutes. Total cell-associated radioactivity and acid-resistant (internalized) radioactivity are determined by gamma- or beta-counting, as appropriate for the isotope of interest.

To examine the subcellular localization of the internalized antibody, cellular organelles are fractionated on Percoll gradients to identify whether the internalized radioactivity targets low density surface membrane fractions or high density lysosomal fractions. Briefly, cells are incubated at 4° C. with saturating concentrations of radiolableled mAbs, washed, and then incubated at 37° C. for 0 to 24 h. Cell aliquots (50×10⁶ cells) are then suspended in TES buffer (10 mM triethanolamine, pH 7.5), disrupted using a Dounce homogenizer, and sedimented at 250×g to remove nuclei and unbroken cells. Supernatant (1 ml) are layered on the surface of a 20% solution of Percoll in TES buffer (9 ml) and centrifuged at 4° C. for 60 min at 20,000×g. Serial 0.5 ml fractions are collected and assayed for radioactivity and for lysosomal 62 -galactosidase activity.

To examine the degradation and catabolism of radiolabeled antibody, antibody and CD19-positive cells are combined at 37° C. for varying periods of time (e.g., 0, 2, 4, 8, 28, 48, and 72 hr), and culture supernatant (0.2 ml) is mixed with 0.5 ml 25% trichloroacetic acid (TCA) to precipitate protein-bound radioactivity released from the cells. Precipitates are then washed with 0.5 ml 25% TCA, and the radioactivity in the pellets (TCA-insoluble) and supernatants (TCA-soluble) is determined. The TCA-insoluble portion represents the labeled antibody conjugate shed in intact form into supernatant and the TCA-soluble portion represents protein-free radioactivity that has been metabolized and excreted by tumor cells.

In vitro cytotoxicity can be readily examined for antibodies labeled with alpha particle-emitting isotopes such as ²²⁵Ac and ²¹³Bi as described (Nikula et al., J. Nucl. Med. 40:166, 1999; McDevitt et al., supra, 2001). 50,000 target cells (CD19-positive cells such as Ramos and CD19-negative control cells such as Molt-4) per well are treated with ²²⁵Ac and ²¹³Bi labeled constructs in 96 well plates at 37° C. in 5% CO₂ for 24-96 hours, at which time cell viability is assessed by MTT assay and/or uptake of H-3 thymidine assay. Cytotoxicity is expressed relative to that seen with 1 M HCl (100% cell kill) and media (background cell kill). Specificity is determined by use of control cells, control radioconjugates, and excess unlabeled anti-CD19 antibody. The effects of antibody concentration, specific activity, activity concentration, and time of exposure can be assessed. Cell killing at various specific activities can then be correlated with total, surface and internalized nuclides as well as the metabolism and intracellular localization of the anti-CD19 conjugates. LD₅₀ values are calculated by plotting cell viability as a function of the number of ²²⁵Ac and/or ²¹³Bi atoms bound on the cells.

The radionuclide decay of ²²⁵Ac yields two daughter radionuclides, ²²¹Fr and ²¹³Bi, that can be monitored by gamma spectroscopy as described by McDevitt et al., supra, 2001.

### EXAMPLE 3

### In Vivo Activity of Anti-CD19 Antibodies Against B Cell Malignancies

A number animal models of human B-cell lymphoma have been developed for evaluation of immunotherapeutic agents (Ghetie et al., Int. J. Cancer, 45:481, 1990; Shah et al., Cancer Res., 53:1360, 1993). These include both disseminated and solid tumor models generated following i.v. and i.m. inoculation of SCID mice with human lymphoma cell lines, such as Ramos.

One solid tumor model employs Ramos cells. Female SCID mice, weighing 18-24 grams, are purchased from Taconic Laboratories (Germantown, N.Y.) or other source. Mice are injected with 10⁶-10⁷ Ramos tumor cells intramuscularly in the hind flank. When the tumor reaches a pre-determined size (approximately 1 cm²), the mice are treated with anti-CD 19 or control antibodies that are either radiolabeled or unlabeled as above. Doses may range to ˜10 mCi/kg or higher for ⁹⁰Y-labeled or ²¹³Bi-labeled antibodies, although the optimal dose must be determined empirically in each case. Groups of the animals are treated with single or multiple doses of drug. The health of the animals is monitored daily or more frequently. The mice are terminated when they appear severely ill or when tumor size exceeds approximately 3 cm². Statistical differences between therapy groups is determined from the data as analyzed using an analysis of variance (ANOVA) method, and animal survival data will be illustrated using Kaplan-Meier plots. Typically, p values of less than 0.05 are considered to be significant.

A disseminated tumor model employs the Daudi human B cell line. Female SCID mice, weighing 18-24 grams, are purchased from Taconic Laboratories (Germantown, N.Y.) or other source. Mice are injected with 10⁶-10⁷ Daudi tumor cells intravenously via the tail vein. Starting approximately 24 hours post-injection, the animals are treated with one or more doses of radiolabeled antibody. Doses may range to ˜10 mCi/kg or higher for ⁹⁰Y-labeled or ²¹³Bi-labeled antibodies, although the optimal dose must be determined empirically in each case. The health of the animals is monitored daily or more frequently, and the animals are euthanized when they become severely ill. Statistical differences between therapy groups are determined from the data as analyzed using an analysis of variance (ANOVA) method, and animal survival data will be illustrated using Kaplan-Meier plots. Typically, p values of less than 0.05 are considered to be significant.

The tumor models can be modified to test whether delivery of radiolabeled mAb to tumor can be improved by predosing with unlabeled mAb. SCID mice bearing lymphoma xenografts are injected with radiolabeled anti-CD19 antibody (typically <1 μg) with or without a prior single injection of unlabeled antibody (typically 5-100 μg). Several days later, animals are sacrificed for evaluation of the distribution of radioactivity in the tumor, normal tissue, and blood. If predosing with unlabeled mAb improves delivery and targeting of radiolabeled mAb to the xenografts, this approach can be applied and optimized in further preclinical and clinical studies.

Dose-ranging studies is performed to determine the toxicity of the radiolabeled antibodies when administered via intravenous or other routes to normal and tumor-bearing mice. The animals are monitored for physical appearance, weight change, tumor size, and survival rate. Animals are sacrificed during and at the conclusion of the study in order to collect blood and body tissues for histopathology and evaluation.

### EXAMPLE 4

### Use of ²²⁵Ac-Anti-CD19 Antibodies Against B Cell Malignancies

Methods

Construct preparation

The radiolabeled [²²⁵Ac]DOTA-IgG complexes that are used in these studies are prepared using a two-step labeling method. The anti-CD19 antibodies that are used include: B4, HD37, BU12, 4G7, J4.119, B43, SJ25C1, and CLB-CD19. A two-step labeling method is used that allows mCi amounts of ²²⁵Ac (and ¹⁷⁷Lu, ¹¹¹In) labeled DOTA-SCN species to be prepared at pH 4.5-5 using 2 M acetate buffer at 55° to 60° C. for 30 min in high yield. Subsequently, the [²²⁵Ac]DOTA-SCN is mixed with IgG with 1 M carbonate buffer to adjust the pH to 8.5-9 at 37° C. for 30 min. The final product is purified by size exclusion chromatography using a 10-ml BioRad 10DG column and 1% human serum albumin (HAS). Typical reaction provide sufficient amounts of stable ²²⁵Ac labeled drug for these studies. Constructs thus prepared are assayed using established ITLC methods that quantify labeled IgG, free [²²⁵Ac]chelate and unbound ²²⁵Ac, and cell-based immunoreactivity assays [Nikula et al., J. Nucl. Med. 40, 166-176 (1999)].

Evaluation of Two Different DOTAs Using the Two-step Labeling Process:

Two different DOTA molecules are evaluated for preparation of ²²⁵Ac-anti-CD19 antibody constructs, as described above: MeO-DOTA-NCS, [(5-isothiocyanato-2-methoxyphenyl)-1,4,7,10-tetraazacyclododecane-1,4,7,10-tetraacetic acid], CAS registry number 130707-79-8; and 2B-DOTA, [2-(p-isothiocyanatobenzyl)-1,4,7,10-tetraazacyclododecane-1,4,7,10-tetraacetic acid], CAS registry number 127985-74-4. No significant differences in the chemistry, stability, or biodistribution of these Ac-chelates is observed.

Assessment of In Vitro Stability

The stability in vitro of similarly prepared [²²⁵Ac]DOTA-anti-CD19 and [¹⁷⁷Lu]DOTA-anti-CD19 constructs is determined in 100% human serum (Sigma Chemical Co., St. Louis, Mo.), 100% mouse serum, and 25% human serum albumin (Swiss Red Cross, Bern, Switzerland) at 37° C. for 15 days. A 0.20 ml aliquot of either [²²⁵Ac]anti-CD19 or [¹⁷⁷Lu]anti-CD19 is added to 4.0 ml of each of the three media. At successive time points, 0.05 ml is removed from the six samples and mixed with 0.01 ml of 10 mM diethylenetriaminepentaacetic acid (DTPA) (Aldrich Chemical Co., Milwaukee, Wis.) for 15 min. at 37° C. After this 15 min incubation period, an aliquot is removed and spotted on instant thin-layer chromatography paper impregnated with silica gel (Gelman Science Inc., Ann Arbor, Mich.) and developed with a 0.01 M EDTA solution (triplicate analysis). Strips are dried and counted 4 days later with a gas ionization detector (Ambis 4000, Ambis Inc., San Diego, Calif.). The methods and values for the same two constructs in 100% mouse serum and 25% human serum albumin are substantially identical to those in 100% human serum.

Assessment of In Vivo Stability

In vivo stability is determined by injecting 10 female nude mice (Taconic, Germantown, N.Y.) via tail vein i.v. route with 300 nCi in 0.12 ml of [²²⁵Ac]DOTA-anti-CD19. The purpose is to determine the percentage of ²²⁵Ac that is bound to the anti-CD19 in the mouse serum as a function of time. IgG-bound ²²⁵Ac is determined using a Protein A Sepharose CL-48 (Amersham Pharmacia Biotech) precipitation assay. Results are also confirmed using High Performance Liquid Chromatography (HPLC). The HPLC analyses are carried out using a Rainen HPLX system (Rainen, Woburn, Mass.) equipped with a Bioscan Flowcount (Bioscan Inc., Washington, D.C.). The stationary phase is a 300 mm 7.8 mm TSK 3000SWXL size exclusion column (Supelco, Bellefonte, Pa.) and the mobile phase is 0.15 M sodium chloride/0.02 M sodium acetate, pH 6.5. Fractions are collected by hand and counted with a Beckman LS 6000IC beta scintillation counter (Beckman Instruments, Inc., Fullerton, Calif.). In addition, the immunoreactive fraction of ²²⁵Ac-anti-CD19 in the serum is determined using a cell-based assay.

HPLC analysis of the ²²⁵Ac species in the serum also indicates that it is associated with the anti-CD19 IgG and does not transchelate to other serum proteins based upon the observed retention time of the component in the serum samples compared with a sample of the original drug injected. The ²²⁵Ac that is bound to anti-CD19 remains associated with the IgG following injection into a mouse over a 5-day period, demonstrating the stability of the drug in vivo.

Determination of Internalized Radionuclides

Methods for determining the amount of internalized radionuclides are as follows. The assay is performed in the presence of 2% human serum. B cells are treated with [²²⁵Ac]bound to anti-CD19 antibody (e.g., B4, HD37, BU12, 4G7, J4.119, B43, SJ25C1, or CLB-CD19 (antibody-to-antigen excess) for 90 min, pelleted and washed 3× with ice-cold PBS and then resuspended in fresh media for a period of 5 hours at 37° C. After this 5 hours incubation the cells are pelleted, washed 3× with ice-cold PBS. The outside surface-bound [²²⁵Ac]anti-CD19 antibody is stripped from the pelleted cells with 1 ml 50 mM glycine (Aldrich Chemical Co., Inc., Milwaukee, Wis.)/50 mM NaCl (Aldrich Chemical Co, Inc.), pH 2.8, at 24° C. for 10 min. The composition of the surface-bound and internalized radioactivity are determined by counting the samples repeatedly at different times with a Packard Cobra Gamma Counter (Packard Instrument Co., Inc., Meriden, Conn.) using two energy windows (²²¹Fr in a 185-250 keV window and ²¹³Bi in a 360-480 keV window).

Equivalents

All references disclosed herein are incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for treating a B cell malignancy in a subject comprising
administering to a subject in need of such treatment an amount of a composition comprising an anti-CD19 immunotoxin and a pharmaceutically acceptable carrier effective to treat the B cell malignancy.

2. The method of claim 1, wherein the anti-CD19 immunotoxin is labeled with a cytotoxic radionuclide or radiotherapeutic isotope.

3. The method of claim 2, wherein the cytotoxic radionuclide or radiotherapeutic isotope is an alpha-emitting isotope.

4. The method of claim 3, wherein the alpha-emitting isotope is selected from the group consisting of ²²⁵Ac, ²¹¹At, ²¹²Bi, ²¹³Bi, ²¹²Pb, ²²⁴Ra, and ²²³Ra.

5. The method of claim 2, wherein the cytotoxic radionuclide or radiotherapeutic isotope is a beta-emitting isotope.

6. The method of claim 5, wherein the beta-emitting isotope is selected from the group consisting of ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, and ⁶⁴Cu.

7. The method of claim 2, wherein the cytotoxic radionuclide or radiotherapeutic isotope emits Auger and low energy electrons.

8. The method of claim 7, wherein the isotope that emits Auger and low energy electrons is selected from the group consisting of ¹²⁵I, ¹²³I and ⁷⁷Br.

9. The method of claim 1, wherein the composition is administered intravenously.

10. The method of claim 1, wherein the amount of the anti-CD19 immunotoxin administered to the subject is between about 10 μg/kg and about 100,000 μg/kg.

11. The method of claim 10, wherein the amount of the anti-CD19 immunotoxin administered to the subject is between about 100 μg/kg and about 10,000 μg/kg.

12. The method of claim 1, wherein the anti-CD19 immunotoxin includes a radionuclide and wherein the amount of the radionuclide administered to the subject is between about 0.001 mCi/kg and about 10 mCi/kg.

13. The method of claim 12, wherein the amount of the radionuclide administered to the subject is between about 0.1 mCi/kg and about 1.0 mCi/kg.

14. The method of claim 12, wherein the amount of the radionuclide administered to the subject is between about 0.005 mCi/kg and about 0.1 mCi/kg.

15. The method of claim 1, wherein the anti-CD19 immunotoxin comprises a monoclonal anti-CD19 antibody or antigen-binding fragment thereof.

16. The method of claim 15, wherein the monoclonal anti-CD19 antibody is a human monoclonal antibody.

17. The method of claim 15, wherein the monoclonal anti-CD19 antibody is a humanized monoclonal antibody.

18. The method of claim 15, wherein the monoclonal anti-CD19 antibody is selected from the group consisting of B4, HD37, BU12, 4G7, J4.119, B43, SJ25C1, and CLB-CD19.

19. The method of claim 1, wherein the B cell malignancy is selected from the group consisting of B cell non-Hodgkin's lymphoma (NHL), B cell acute lymphocytic leukemia (B-ALL), B cell precursor acute lymphocytic leukemia (pre-B-ALL), B cell chronic lymphocytic leukemia (B-CLL) and hairy cell leukemia.

20. The method of claim 1, wherein the B cell malignancy comprises B cells that do not express CD20.

21. The method of claim 1, further comprising administering to the subject one or more immunomodulatory agents.

22. The method of claim 21, wherein the immunomodulatory agent is a cytokine or an adjuvant.

23. The method of claim 22, wherein the cytokine is selected from the group consisting of interleukin-1 (IL-1), IL-2, IL-3, IL-12, IL-15, IL-18, G-CSF, GM-CSF, thrombopoietin, and γ-interferon.

24. The method of claim 1, wherein the anti-CD19 immunotoxin is labeled with a chemical toxin or chemotherapeutic agent.

25. The method of claim 24, wherein the chemical toxin or chemotherapeutic agent is selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

26. The method of claim 1, wherein the anti-CD19 immunotoxin is labeled with an agent that acts on the tumor neovasculature or an immunomodulator.

27. The method of claim 26, wherein the agent that acts on the tumor neovasculature is selected from the group consisting of combrestatin A4, angiostatin and endostatin.

28. The method of claim 26, wherein the immunomodulator is selected from the group consisting of α-interferon, γ-interferon, and tumor necrosis factor alpha (TNFα).

29. A composition comprising an anti-CD19 immunotoxin and a pharmaceutically acceptable carrier, wherein the anti-CD19 immunotoxin is labeled with a cytotoxic radionuclide or radiotherapeutic isotope.

30. The composition of claim 29, wherein the cytotoxic radionuclide or radiotherapeutic isotope is an alpha-emitting isotope.

31. The composition of claim 30, wherein the alpha-emitting isotope is selected from the group consisting of ²²⁵Ac, ²¹¹At, ²¹²Bi, ²¹³Bi, ²¹²Pb, ²²⁴Ra, and ²²³Ra.

32. The composition of claim 29, wherein the cytotoxic radionuclide or radiotherapeutic isotope is a beta-emitting isotope.

33. The composition of claim 32, wherein the beta-emitting isotope is selected from the group consisting of ¹⁸⁶Re ¹⁸⁸Re, ⁹⁰Y, ¹³¹I, ⁶⁷Cu, ¹⁷⁷Lu, ¹⁵³Sm, ⁶⁶Ho, and ⁶⁴Cu.

34. The composition of claim 29, wherein the cytotoxic radionuclide or radiotherapeutic isotope emits Auger and low energy electrons.

35. The composition of claim 34, wherein the isotope that emits Auger and low energy electrons is selected from the group consisting of ¹²⁵I, ¹²³I and ⁷⁷Br.

36. The composition of claim 29, wherein the composition is formulated for intravenous administration.

37. The composition of claim 29, wherein the anti-CD19 immunotoxin comprises a monoclonal anti-CD19 antibody or antigen-binding fragment thereof.

38. The composition of claim 37, wherein the monoclonal anti-CD19 antibody is a human monoclonal antibody.

39. The composition of claim 37, wherein the monoclonal anti-CD19 antibody is a humanized monoclonal antibody.

40. The composition of claim 37, wherein the monoclonal anti-CD19 antibody is selected from the group consisting of B4, HD37, BU12, 4G7, J4.119, B43, SJ25C1, and CLB-CD19.

41. The composition of claim 29, further comprising one or more immunomodulatory agents.

42. The composition of claim 41, wherein the immunomodulatory agent is a cytokine or an adjuvant.

43. The composition of claim 42, wherein the cytokine is selected from the group consisting of interleukin-1 (IL-1), IL-2, IL-3, IL-12, IL-15, IL-18, G-CSF, GM-CSF, thrombopoietin, and γ-interferon.

44. A composition comprising an anti-CD19 immunotoxin and a pharmaceutically acceptable carrier, wherein the anti-CD19 immunotoxin is labeled with a chemical toxin or chemotherapeutic agent.

45. The composition of claim 44, wherein the chemical toxin or chemotherapeutic agent is selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

46. The composition of claim 44, wherein the anti-CD19 immunotoxin is labeled with an agent that acts on the tumor neovasculature or an immunomodulator.

47. The composition of claim 46, wherein the agent that acts on the tumor neovasculature is selected from the group consisting of combrestatin A4, angiostatin and endostatin.

48. The composition of claim 46, wherein the immunomodulator is selected from the group consisting of α-interferon, γ-interferon, and tumor necrosis factor alpha (TNFα).

49. The composition of claim 44, further comprising one or more immunomodulatory agents.

50. The composition of claim 49, wherein the immunomodulatory agent is a cytokine or an adjuvant.

51. The composition of claim 50, wherein the cytokine is selected from the group consisting of interleukin-1 (IL-1), IL-2, IL-3, IL-12, IL-15, IL-18, G-CSF, GM-CSF, thrombopoietin, and γ-interferon.

52. An anti-CD19 immunotoxin comprising an anti-CD19 antibody or antigen binding fragment thereof labeled with a cytotoxic radionuclide or radiotherapeutic isotope.

53. The immunotoxin of claim 52, wherein the cytotoxic radionuclide or radiotherapeutic isotope is an alpha-emitting isotope.

54. The immunotoxin of claim 53, wherein the alpha-emitting isotope is selected from the group consisting of ²²⁵Ac, ²¹¹At, ²¹²Bi, ²¹³Bi, ²¹²Pb ²²⁴Ra, and ²²³Ra.

55. The immunotoxin of claim 52, wherein the cytotoxic radionuclide or radiotherapeutic isotope is a beta-emitting isotope.

56. The immunotoxin of claim 55, wherein the beta-emitting isotope is selected from the group consisting of ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, and ⁶⁴Cu.

57. The immunotoxin of claim 52, wherein the cytotoxic radionuclide or radiotherapeutic isotope emits Auger and low energy electrons.

58. The immunotoxin of claim 57, wherein the isotope that emits Auger and low energy electrons is selected from the group consisting of ¹²⁵I, ¹²³I and ⁷⁷Br.

59. The immunotoxin of claim 52, wherein the anti-CD19 immunotoxin comprises a monoclonal anti-CD19 antibody or antigen-binding fragment thereof.

60. The immunotoxin of claim 59, wherein the monoclonal anti-CD19 antibody is a human monoclonal antibody.

61. The immunotoxin of claim 59, wherein the monoclonal anti-CD19 antibody is a humanized monoclonal antibody.

62. The immunotoxin of claim 59, wherein the monoclonal anti-CD19 antibody is selected from the group consisting of B4, HD37, BU12, 4G7, J4.119, B43, SJ25C1, and CLB-CD19.

63. An anti-CD19 immunotoxin comprising an anti-CD19 antibody or antigen binding fragment thereof labeled with a chemical toxin or chemotherapeutic agent.

64. The immunotoxin of claim 63, wherein the chemical toxin or chemotherapeutic agent is selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

65. The immunotoxin of claim 63, wherein the anti-CD19 immunotoxin comprises a monoclonal anti-CD19 antibody or antigen-binding fragment thereof.

66. The immunotoxin of claim 65, wherein the monoclonal anti-CD19 antibody is a human monoclonal antibody.

67. The immunotoxin of claim 65, wherein the monoclonal anti-CD19 antibody is a humanized monoclonal antibody.

68. The immunotoxin of claim 65, wherein the monoclonal anti-CD19 antibody is selected from the group consisting of B4, HD37, BU12, 4G7, J4.119, B43, SJ25C1, and CLB-CD19.

69. An anti-CD19 immunotoxin comprising an anti-CD19 antibody or antigen binding fragment thereof labeled with an agent that acts on the tumor neovasculature or an immunomodulator.

70. The immunotoxin of claim 69, wherein the agent that acts on the tumor neovasculature is selected from the group consisting of combrestatin A4, angiostatin and endostatin.

71. The immunotoxin of claim 69, wherein the immunomodulator is selected from the group consisting of α-interferon, γ-interferon, and tumor necrosis factor alpha (TNFα).

72. The immunotoxin of claim 69, wherein the anti-CD19 immunotoxin comprises a monoclonal anti-CD19 antibody or antigen-binding fragment thereof.

73. The immunotoxin of claim 72, wherein the monoclonal anti-CD19 antibody is a human monoclonal antibody.

74. The immunotoxin of claim 72, wherein the monoclonal anti-CD19 antibody is a humanized monoclonal antibody.

75. The immunotoxin of claim 72, wherein the monoclonal anti-CD19 antibody is selected from the group consisting of B4, HD37, BU12, 4G7, J4.119, B43, SJ25C1, and CLB-CD19.

76. A method for treating an autoimmune disorder in a subject comprising
administering to a subject in need of such treatment an amount of a composition comprising an anti-CD19 immunotoxin of any one of claims 52-75 and a pharmaceutically acceptable carrier, said amount effective to treat the autoimmune disorder.

77. The method of claim 76, wherein the autoimmune disorder is selected from the group consisting of plasma cell disorders including IgM polyneuropathies, immune thrombocytopenias, and autoimmune hemolytic anemias; Sjogren's syndrome; multiple sclerosis; rheumatoid arthritis; autoimmune lymphoproliferative syndrome (ALPS); sarcoidosis; diabetes; systemic lupus erythematosus; and bullous pemphigoid.

78. A method for depleting or reducing the number of CD19+ B cells in a subject comprising
administering to a subject in need of such treatment an amount of a composition comprising an anti-CD19 immunotoxin of any one of claims 52-75 and a pharmaceutically acceptable carrier, the amount effective to deplete or reduce the number of CD19+ B cells.

79. The method of claim 78 wherein the composition is administered before, during or after implantation of a xenograft or a donor organ or tissue transplant.

80. The method of claim 79, wherein the effective amount prevents or reduces deleterious antibody formation.

81. The method of claim 80, wherein the deleterious antibody is an autoantibody, a xenograft antibody, or an anti-transplant antibody.

82. Use of a composition comprising an anti-CD19 immunotoxin for the manufacture of a medicament for treating a B cell malignancy.

83. The use of claim 82, wherein the anti-CD19 immunotoxin is labeled with a cytotoxic radionuclide or radiotherapeutic isotope.

84. The use of claim 83, wherein the cytotoxic radionuclide or radiotherapeutic isotope is an alpha-emitting isotope.

85. The use of claim 84, wherein the alpha-emitting isotope is selected from the group consisting of ²²⁵Ac, ²¹¹At, ²¹²Bi, ²¹³Bi, ²¹²Pb ²²⁴Ra, and ²²³Ra.

86. The use of claim 83, wherein the cytotoxic radionuclide or radiotherapeutic isotope is a beta-emitting isotope.

87. The use of claim 86, wherein the beta-emitting isotope is selected from the group consisting of ¹⁸⁶Re, ¹⁸⁸Re, ⁹⁰Y, ¹³¹I, ⁶⁷Cu, ¹⁷⁷Lu, ¹⁵³Sm, ¹⁶⁶Ho, and ⁶⁴Cu.

88. The use of claim 83, wherein the cytotoxic radionuclide or radiotherapeutic isotope emits Auger and low energy electrons.

89. The use of claim 88, wherein the isotope that emits Auger and low energy electrons is selected from the group consisting of ¹²⁵I, ¹²³I and ⁷⁷Br.

90. The use of claim 82, wherein the medicament is suitable for intraveneous administration.

91. The use of claim 82, wherein the medicament is suitable to provide immunotoxin between about 10 μg/kg and about 100,000 μg/kg to a subject.

92. The use of claim 91, wherein the composition contains an amount of the anti-CD19 immunotoxin suitable for administration to the subject at a concentration between about 100 μg/kg and about 10,000 μg/kg.

93. The use of claim 82, wherein the anti-CD19 immunotoxin includes a radionuclide and wherein the medicament is suitable to provide an amount of the radionuclide between about 0.001 mCi/kg and about 10 mCi/kg to a subject.

94. The use of claim 93, wherein the medicament is suitable to provide between about 0.1 mCi/kg and about 1.0 mCi/kg to a subject.

95. The use of claim 93, wherein the medicament is suitable to provide between about 0.005 mCi/kg and about 0.1 mCi/kg to a subject.

96. The use of claim 82, wherein the anti-CD19 immunotoxin comprises a monoclonal anti-CD19 antibody or antigen-binding fragment thereof.

97. The use of claim 96, wherein the monoclonal anti-CD19 antibody is a human monoclonal antibody.

98. The use of claim 96, wherein the monoclonal anti-CD19 antibody is a humanized monoclonal antibody.

99. The use of claim 96, wherein the monoclonal anti-CD19 antibody is selected from the group consisting of B4, HD37, BU12, 4G7, J4.119, B43, SJ25C1, and CLB-CD19.

100. The use of claim 82, wherein the B cell malignancy is selected from the group consisting of B cell non-Hodgkin's lymphoma (NHL), B cell acute lymphocytic leukemia (B-ALL), B cell precursor acute lymphocytic leukemia (pre-B-ALL), B cell chronic lymphocytic leukemia (B-CLL) and hairy cell leukemia.

101. The use of claim 82, wherein the B cell malignancy comprises B cells that do not express CD20.

102. The use of claim 82, wherein the medicament further comprises one or more immunomodulatdry agents.

103. The use of claim 102, wherein the immunomodulatory agent is a cytokine or an adjuvant.

104. The use of claim 103, wherein the cytokine is selected from the group consisting of interleukin-1 (IL-1), IL-2, IL-3, IL-12, IL-15, IL-18, G-CSF, GM-CSF, thrombopoietin, and γ-interferon.

105. The use of claim 82, wherein the anti-CD19 immunotoxin is labeled with a chemical toxin or chemotherapeutic agent.

106. The use of claim 105, wherein the chemical toxin or chemotherapeutic agent is selected from the group consisting of an enediyne such as calicheamicin and esperamicin; duocarmycin, methotrexate, doxorubicin, melphalan, chlorambucil, ARA-C, vindesine, mitomycin C, cis-platinum, etoposide, bleomycin and 5-fluorouracil.

107. The use of claim 82, wherein the anti-CD19 immunotoxin is labeled with an agent that acts on the tumor neovasculature or an immunomodulator.

108. The use of claim 107, wherein the agent that acts on the tumor neovasculature is selected from the group consisting of combrestatin A4, angiostatin and endostatin.

109. The use of claim 107, wherein the immunomodulator is selected from the group consisting of α-interferon, γ-interferon, and tumor necrosis factor alpha (TNFα).

110. Use of a composition comprising an anti-CD19 immunotoxin of any one of claims 52-75 for the manufacture of a medicament for treating an autoimmune disorder in a subject.

111. The use of claim 110, wherein the autoimmune disorder is selected from the group consisting of plasma cell disorders including IgM polyneuropathies, immune thrombocytopenias, and autoimmune hemolytic anemias; Sjogren's syndrome; multiple sclerosis; rheumatoid arthritis; autoimmune lymphoproliferative syndrome (ALPS); sarcoidosis; diabetes; systemic lupus erythematosus; and bullous pemphigoid.

112. Use of a composition comprising an anti-CD19 immunotoxin of any one of claims 52-75 for the manufacture of a medicament to deplete or reduce the number of CD19+ B cells in a subject.

113. The use of claim 112 wherein the composition is suitable for administration before, during or after implantation of a xenograft or a donor organ or tissue transplant.

114. The use of claim 113, wherein the medicament prevents or reduces deleterious antibody formation.

115. The use of claim 114, wherein the deleterious antibody is an autoantibody, a xenograft antibody, or an anti-transplant antibody.
